(19) United States
(12) Patent Application Publication
(10) Pub. No.: US 2010/0082071 A1
(43) Pub. Date: Apr. 1, 2010

(54) Composite Screw Having A Metallic Pin and a Polymeric Thread

(76) Inventor: Missoum Moumene, Newton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/239,616

(22) Filed: Sep. 26, 2008

(51) Int. Cl. A61B 17/86

(52) U.S. Cl. 606/318; 606/309

(57) ABSTRACT

A composite screw preferably made of a metallic pin embedded in a polymeric tubular structure having the outwardly facing threads, wherein the metallic pin comprises a screw head, an intermediate shaft and a screw tip, and the polymeric threaded tube is either molded onto the metallic shaft or the threaded is machined after molding a polymeric structure onto the shaft.

### BACKGROUND OF THE INVENTION

Pedicle screws used in spinal surgery are disclosed, for example, in U.S. Pat. No. 5,672,176.

Spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. The fixation rods can have a predetermined contour that has been designed according to the properties of the target implantation site, and once installed, the instrument holds the vertebrae in a desired spatial relationship, either until desired healing or spinal fusion has taken place, or for some longer period of time.

Spinal fixation devices can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a bone screw with a threaded shank that is adapted to be threaded into a vertebra, and a rod-receiving element, usually in the form of a U-shaped slot formed in the head. The shank and rod-receiving assembly can be provided as a monoaxial screw, whereby the rod-receiving element is fixed with respect to the shank, or a polyaxial screw, whereby the rod-receiving element has free angular movement with respect to the shank. In use, the shank portion of each screw is threaded into a vertebra, and once properly positioned, a fixation rod is seated into the rod-receiving element of each screw. The rod is then locked in place by tightening a set-screw, plug, or similar type of fastening mechanism into the rod-receiving element.

Polyaxial pedicle screws such as disclosed in Biedermann's U.S. Pat. No. 5,443,467, incorporated herein by reference, are used for connecting vertebrae to rods in spinal surgery. They incorporate a ball joint at the connection to the rod to allow the surgeon some flexibility in placing the screws. Tightening a nut on the screw compresses the ball joint components to lock the angular position of the ball joint.

Pedicle screws are typically much stiffer than the surrounding bone and more specifically much stiffer than the interior cancellous region of a vertebra body into which the screw is inserted. This mismatch in stiffness may cause fracture in the surrounding cancellous vertebra body bone, allowing the screw to undesirably toggle (like a windshield wiper) within the vertebra body. This toggling ultimately leads to screw loosening and failure of the construct. These issues are of particular concern in osteoporotic bone and aging spines.

Gefen, “Computational simulation of stress shielding and bone resorption around existing and computer designed orthopaedic screws”, Medical and Biological Engineering & Computing, 2002, Vol. 40, pp. 311-322, has reported that conventional bone screws produce sufficient stress shielding so as to cause bone loss around the distal tip of the screw.

U.S. Pat. No. 3,384,141 (Kost) describes a composite screw having a metallic pin surrounded by a plastic thread.

### SUMMARY OF THE INVENTION

This invention provides a composite pedicle screw. The composite screw (FIG. 1) is preferably made of a metallic pin (FIG. 2) embedded in a polymeric tubular structure having the outwardly facing threads (FIG. 3). The metallic pin preferably consists of a screw head, an intermediate shaft and an insertion tip. The polymeric tube is typically either molded on the metallic shaft in a threaded form, or is machined to form the thread after molding the blank polymeric structure on the shaft.

In preferred embodiments, this invention provides a solution to the above-mentioned stiffness mismatch problem by gradually reducing the thickness (and therefore the stiffness) of the shaft from the screw head to the distal tip of the screw. This narrowing shaft region of the screw corresponds to the cancellous region of the vertebral body. Thus, the region of the screw tip will have stiffness comparable to that of vertebra body cancellous bone, while the region of the screw in the pedicle will have stiffness substantially equivalent to that of the (cortical) vertebra pedicle. Typically, no modification need be made to the conventional metallic head. The shaft can be tapered from the tip of the screw to the head of the screw using one or multiple tapered sections.

Therefore, in accordance with the present invention, there is provided a composite bone fixation system comprising:
i) a pin comprising:
   a) a proximal head,
   b) a distal tip, and
   c) an intermediate shaft portion having a distally narrowing outer surface,
ii) a threaded polymeric tube comprising an inner bore and a threaded outer surface,
   wherein the threaded polymeric tube is disposed over the outer surface of the intermediate shaft portion.

The metallic shaft can be textured for increased adhesion of polymeric tube thereto. The polymer can be selected from any biocompatible polymer (and is preferably PEEK) and may be reinforced by fibers. The metallic shaft can be made of any biocompatible metal, and preferably comprises titanium.

In preferred embodiments, the composite screw has a variable stiffness along the screw shaft from the tip to the head. This is typically accomplished by reducing its thickness. Preferably, the composite screw is more flexible than the typical metal screw, yet possesses a conventional metallic head. Typically, the composite screw has three principal sections possessing different stiffnesses: a) a distal section composed primarily of polymer and having stiffness similar to that of cancellous bone, b) a middle section composed of both polymer and metal with an overall stiffness similar to that of cortical bone, and c) a proximal metallic section having the neck and the head of the screw. The composite screw will preferably reduce or eliminate the screw toggling and therefore screw loosening. This is viewed as having particular advantage for osteoporotic bones. The composite screw is meant to reduce the stiffness of the screw by introducing polymeric thread.

Also in accordance with the present invention, there is provided a composite bone fixation system comprising:
i) a pin comprising:
   a) a proximal head having a first maximum diameter D₁,
   b) a distal tip having a second maximum diameter D₂, and
   c) an intermediate shaft portion having an outer surface defining a third maximum diameter D₃,
ii) a threaded polymeric tube comprising an inner bore and a threaded outer surface,
   wherein the threaded polymeric tube is disposed over the outer surface of the substantially cylindrical intermediate shaft portion, and
   wherein each of the first maximum diameter D₁ and second maximum diameter D₂ is greater than the third maximum diameter D₃.

### DESCRIPTION OF THE FIGURES

FIG. 1 is a perspective view of the composite bone fixation system of the present invention.

FIG. 2 is a perspective view of the pin component of FIG. 1.

FIG. 3 is a perspective view of the tubular component of FIG. 1.

FIG. 4 is a cross-section of a composite bone fixation system of the present invention, wherein the pin has a distally narrowing shaft.

### DETAILED DESCRIPTION OF THE INVENTION

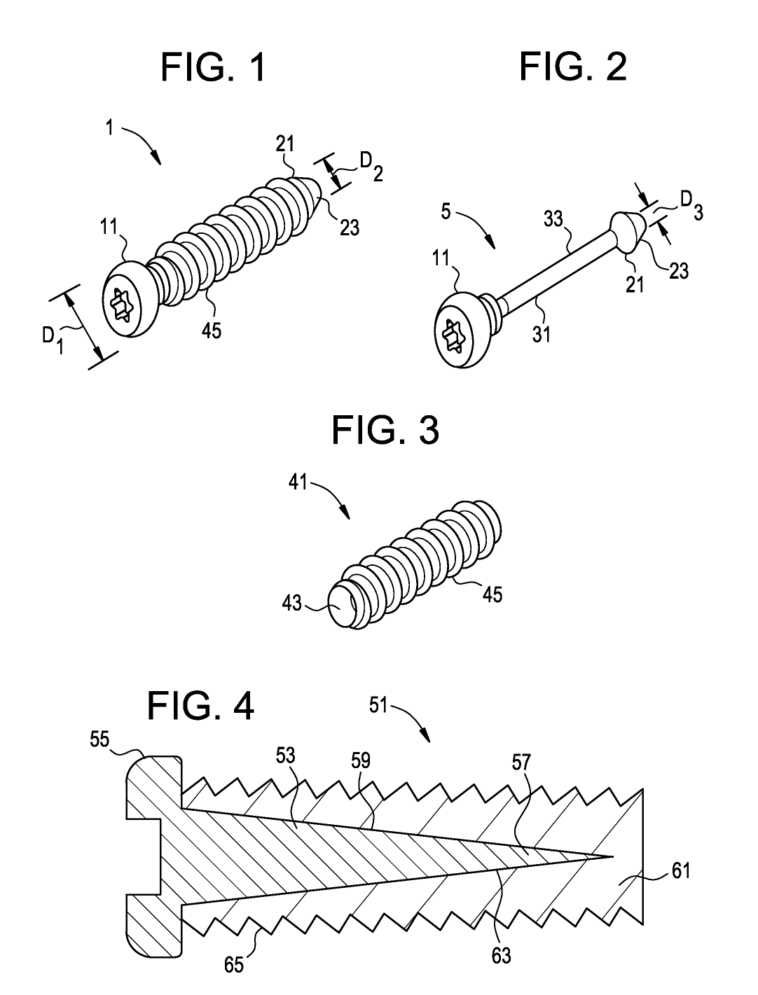

Referring now to FIGS. 1-3, there is provided a composite bone fixation system 1 comprising:
i) a pin 5 comprising:
   a) a proximal head 11 having a first maximum diameter D₁,
   b) a distal tip 21 having a second maximum diameter D₂ and a distally narrowing insertion taper 23, and
   c) a substantially cylindrical intermediate shaft portion 31 having an outer surface 33 defining a third maximum diameter D₃,
ii) a threaded polymeric tube 41 comprising an inner bore 43 and a threaded outer surface 45,
   wherein the threaded polymeric tube wraps around the outer surface of the substantially cylindrical intermediate shaft portion, and
   wherein each of the first maximum diameter D₁ and second maximum diameter D₂ is greater than the third maximum diameter D₃.

When each of the first maximum diameter D₁ and second maximum diameter D₂ is greater than the third maximum diameter D₃, the polymeric tube (which circumferentially contacts the outer surface of the shaft and so also has a bore diameter of essentially D₃) will remain in the middle of the pin because it is prevented by the larger head and tip components from sliding off either end of the pin.

In some embodiments, the distal tip of FIG. 2 comprises a distally narrowing insertion taper. This facilitates insertion of the screw into the vertebral body.

In some embodiments (not shown), the distal tip of FIG. 2 is detachable from the shaft. The detachability helps the assembly of the composite device. In the manufacturing process, the pin having the detached tip is first provided. The polymeric tube is slid onto the shaft of the pin and set in its final place. Once the polymeric tube is in place, the distal tip is attached to the end of the shaft, thereby fixing the position of the tube. In some embodiments thereof, the detachable distal tip and the shaft comprise a male/female connection. In some embodiments thereof, the male/female connection is threaded. In other embodiments thereof, the male/female connection is a taper lock.

Referring now to FIG. 4, there is provided a composite bone fixation system 51 comprising:
i) a pin 53 comprising:
   a) a proximal head 55,
   b) a distal tip 57, and
   c) an intermediate shaft portion 59 having a distally narrowing outer surface,
ii) a threaded polymeric tube 61 comprising an inner bore 63 and a threaded outer surface 65,
   wherein the threaded polymeric tube is disposed over the outer surface of the intermediate shaft portion.

FIG. 4 provides a solution to the above-mentioned stiffness mismatch problem by gradually reducing the thickness (and therefore the stiffness) of the shaft from the screw head to the distal tip of the screw. This narrowing shaft region of the screw corresponds to the cancellous region of the vertebral body. Thus, the region of the screw tip will have stiffness comparable to that of vertebra body cancellous bone, while the region of the screw in the pedicle will have stiffness substantially equivalent to that of the (cortical) vertebra pedicle.

In some embodiments, the distal tip of FIG. 4 is formed by the distal narrowing of the intermediate shaft portion.

In some embodiments, the distal tip of the metallic pin extends to the distal end of the polymeric tube. In other embodiments, as in FIG. 4, the metallic pin terminates prior to the distal end of the polymeric tube.

If a metal is chosen as the material of construction for the pin, then the metal is preferably selected from the group consisting of titanium, titanium alloys (such as Ti-6Al-4V), chrome alloys (such as CrCo or Cr—Co—Mo) and stainless steel.

In some embodiments, the polymer of the threaded polymeric tube is preferably selected from the group consisting of polyesters, (particularly aromatic esters such as polyalkylene terephthalates, polyamides; polyalkenes; poly(vinyl fluoride); PTFE; polyarylethyl ketone PAEK; polyphenylene and mixtures thereof.

In some embodiments, the threaded polymeric tube is a composite comprising carbon fiber. Composites comprising carbon fiber are advantageous in that they typically have a strength and stiffness that is superior to neat polymer materials such as a polyarylethyl ketone PAEK. In some embodiments, each component is made from a polymer composite such as a PEKK-carbon fiber composite.

Preferably, the polymer of the composite tube comprising carbon fiber comprises a polyarylethyl ketone (PAEK). More preferably, the PAEK is selected from the group consisting of polyetherether ketone (PEEK), polyether ketone ketone (PEKK) and polyether ketone (PEK). In preferred embodiments, the PAEK is PEEK.

In some embodiments, the carbon fiber comprises between 1 vol % and 60 vol % (more preferably, between 10 vol % and 50 vol %) of the composite tube. In some embodiments, the polymer and carbon fibers are homogeneously mixed. In others, the material is a laminate. In some embodiments, the carbon fiber is present in a chopped state. Preferably, the chopped carbon fibers have a median length of between 1 mm and 12 mm, more preferably between 4.5 mm and 7.5 mm. In some embodiments, the carbon fiber is present as continuous strands.

In especially preferred embodiments, the composite tube comprises:
a) 40-99% (more preferably, 60-80 vol %) polyarylethyl ketone (PAEK), and
b) 1-60% (more preferably, 20-40 vol %) carbon fiber,
   wherein the polyarylethyl ketone (PAEK) is selected from the group consisting of polyetherether ketone (PEEK), polyether ketone ketone (PEKK) and polyether ketone (PEK).

In some embodiments, the composite tube consists essentially of PAEK and carbon fiber. More preferably, the composite comprises 60-80 wt % PAEK and 20-40 wt % carbon fiber. Still more preferably the composite comprises 65-75 wt % PAEK and 25-35 wt % carbon fiber.

One skilled in the art will appreciate that the screw of the present invention may be configured for use with any type of fixation system—mono-axial or polyaxial. Typically, a bone anchor assembly includes a bone screw, such as a pedicle screw, having a proximal head and a distal bone-engaging portion, which may be an externally threaded screw shank. The bone screw assembly may also have a receiving member that is configured to receive and couple a spinal fixation element, such as a spinal rod or spinal plate, to the bone anchor assembly.

In some embodiments, the bone anchor has a plate and bolt design. The receiving member may be coupled to the bone anchor in any well-known conventional manner. For example, the bone anchor assembly may be poly-axial, as in the present exemplary embodiment in which the bone anchor may be adjustable to multiple angles relative to the receiving member, or the bone anchor assembly may be mono-axial, e.g., the bone anchor is fixed relative to the receiving member. An exemplary poly-axial bone screw is described U.S. Pat. No. 5,672,176, the specification of which is incorporated herein by reference in its entirety. In mono-axial embodiments, the bone anchor and the receiving member may be coaxial or may be oriented at angle with respect to one another. In poly-axial embodiments, the bone anchor may biased to a particular angle or range of angles to provide a favored angle the bone anchor. Exemplary favored-angle bone screws are described in U.S. Patent Application Publication No. 2003/0055426 and U.S. Patent Application Publication No. 2002/0058942, the specifications of which are incorporated herein by reference in their entireties.

Also in accordance with the present invention, there is provided a method of performing spinal surgery, comprising the step of:
a) inserting into a vertebral body a bone screw comprising:

a pin comprising:
i) a proximal head having a first maximum diameter D₁,
ii) a distal tip having a second maximum diameter D₂, and
iii) a substantially cylindrical intermediate shaft portion having an outer surface defining a third maximum diameter D₃,

a threaded polymeric tube comprising an inner bore and a threaded outer surface,

wherein the threaded polymeric tube is disposed over the outer surface of the substantially cylindrical intermediate shaft portion.

We claim:

1. A composite bone fixation system comprising:
i) a pin comprising:
a) a proximal head having a first maximum diameter D₁,
b) a distal tip having a second maximum diameter D₂, and
c) an intermediate shaft portion having an outer surface defining a third maximum diameter D₃,
ii) a threaded polymeric tube comprising an inner bore and a threaded outer surface,
wherein the threaded polymeric tube is disposed over the outer surface of the substantially cylindrical intermediate shaft portion, and
wherein each of the first maximum diameter D₁ and second maximum diameter D₂ is greater than the third maximum diameter D₃.

2. A composite bone fixation system of claim 1 wherein the distal tip comprises a distally narrowing insertion taper.

3. The composite bone fixation system of claim 1 wherein the distal tip is detachable from the shaft.

4. The composite bone fixation system of claim 1 wherein detachable distal tip and the shaft comprise a male/female connection.

5. The composite bone fixation system of claim 1 wherein the male/female connection is threaded.

6. The composite bone fixation system of claim 1 wherein the male/female connection is a taper lock.

7. The composite bone fixation system of claim 1 wherein the pin is metallic.

8. The composite bone fixation system of claim 1 wherein the outer surface of the shaft portion is textured for increased adhesion of polymer

9. The composite bone fixation system of claim 1 wherein the polymeric tube comprises carbon fiber.

10. A method of performing surgery, comprising the steps of:
a) inserting into a vertebral body a screw comprising:
a pin comprising:
j) a proximal head having a first maximum diameter D₁,
ii) a distal tip having a second maximum diameter D₂, and
iii) an intermediate shaft portion having an outer surface defining a third maximum diameter D₃,
a threaded polymeric tube comprising an inner bore and a threaded outer surface,
wherein the threaded polymeric tube is disposed over the outer surface of the intermediate shaft portion.

11. A composite bone fixation system comprising:
i) a pin comprising:
a) a proximal head,
b) a distal tip, and
c) an intermediate shaft portion having a distally narrowing outer surface,
ii) a threaded polymeric tube comprising an inner bore and a threaded outer surface,
wherein the threaded polymeric tube is disposed over the outer surface of the intermediate shaft portion.

12. The composite bone fixation system of claim 11 wherein the distal tip is formed by the distal narrowing of the intermediate shaft portion.

13. The composite bone fixation system of claim 11 wherein the distal tip comprises a distally narrowing insertion taper.

14. The composite bone fixation system of claim 11 wherein the distal tip is detachable from the shaft.

15. The composite bone fixation system of claim 11 wherein detachable distal tip and the shaft comprise a male/female connection.

16. The composite bone fixation system of claim 11 wherein the male/female connection is threaded.

17. The composite bone fixation system of claim 11 wherein the male/female connection is a taper lock.

18. The composite bone fixation system of claim 11 wherein the pin is metallic.

19. The composite bone fixation system of claim 11 wherein the outer surface of the shaft portion is textured for increased adhesion of polymer

20. The composite bone fixation system of claim 11 wherein the polymeric tube comprises carbon fiber.
